(19) United States
(12) Patent Application Publication
(10) Pub. No.: US 2011/0142770 A1
(43) Pub. Date: Jun. 16, 2011

(54) SUNSCREEN COMPOSITIONS INCORPORATING METHYLCELLULOSE AS AN SPF AND/OR PPD BOOSTER AND METHODS

(76) Inventors: Robert B. Fletcher, Midland, MI (US); James A. Gall, Midland, MI (US)

(21) Appl. No.: 12/967,539

(22) Filed: Dec. 14, 2010

Related U.S. Application Data
(60) Provisional application No. 61/287,039, filed on Dec. 16, 2009.

(51) Int. Cl. A61K 8/73; A61Q 17/04

(52) U.S. Cl. 424/59

(57) ABSTRACT

Described are sunscreen compositions, comprising organic pigment particulates and methylcellulose, and methods of boosting the SPF (sun protection factor) and/or PPD (Persistent Pigment Darkening) of a sunscreen composition having organic pigment particulates, comprising including methylcellulose in the sunscreen composition.

This application claims benefit of priority from U.S. Provisional Patent Application No. 61/287,039, filed Dec. 16, 2009, which application is incorporated by reference herein in its entirety.

### FIELD

The present invention relates to sunscreen compositions.

### BACKGROUND

Many countries now require quantifiable UVA protection on sunscreen labels as well as UVB. Achieving today's UVA and UVB label claims can be challenging because high concentrations of sunscreens can be expensive. Accordingly, there is a need for sunscreen boosters which will help achieve high SPF (sun protection factor) and high UVA protection (for example, as measured by the PPD (Persistent Pigment Darkening) method) with lower active concentrations.

### SUMMARY

In one embodiment, the present invention provides sunscreen compositions, comprising organic pigment particulates and methylcellulose.

In another embodiment, the present invention provides methods of boosting the SPF and or PPD of a sunscreen composition having organic pigment particulates, comprising including methylcellulose in the sunscreen composition.

### DETAILED DESCRIPTION

In one embodiment, the present invention provides sunscreen compositions, comprising organic pigment particulates and methylcellulose.

“Organic pigment particulate” refers to carbon-based particles of less than about 250 nm, preferably less than about 200 nm.

In one embodiment, the organic pigment particulates are bisoctrizole. Bisoctrizole (INCI name: methylene bis-benzotriazolyl tetramethylbutylphenol) is a benzotriazole based organic compound which acts as a broad spectrum UV absorber, effective in both UVA and UVB ranges. Bisoctrizole is available from Ciba Specialty Chemicals under the tradename TINOSORB M. In one embodiment, the organic pigment particulates are present in an amount from about 0.1% to about 50% by weight of the composition, and more preferably 1% to about 25%. In a most preferred embodiment, the organic pigment particulates are present in an amount constituting about 10% active.

In one embodiment, the sunscreen compositions of the present inventions further comprise an additional sunscreen agent, wherein the sunscreen agent is at least one of octyl methoxycinnamate, avobenzone, para aminobenzoic acid, homosalate, titanium dioxide, zinc oxide, benzophenones, benzylidenes, salicylates, or other known UV filters.

In one embodiment, the zinc oxide or titanium dioxide are pigment grade. In one embodiment, the zinc oxide or titanium dioxide are transparent zinc oxide or transparent titanium dioxide. Most inorganic metal oxide sunscreen particles used in a sunscreen produce a cosmetically undesirable white appearance caused by light scattering. Thus, the term “transparent,” as used herein has a special meaning, referring to inorganic metal oxide sunscreen particles produced by a variety of processing conditions which render the inorganic metal oxide compositions as clear, or transparent, upon application. In other words, these specially processed inorganic metal oxide compositions do not appear white once applied, hence the moniker of transparent.

Examples of transparent zinc oxide are disclosed in, for example, U.S. Pat. Nos. 5,223,250, 5,372,805, 5,573,753, 5,587,148, and 5,876,688. One example of a transparent zinc oxide is commercially available under the tradename Z-COTE from BASF Corporation (Germany). Another example of transparent zinc oxide is commercially available under the tradename ZINCLEAR IM from Antaria Limited (Australia). Another example of transparent zinc oxide is commercially available under the tradename Z-CLEAR from Actifirm (USA).

Examples of transparent titanium dioxide are disclosed in, for example, U.S. Pat. Nos. 5,573,753, 5,733,895, and 7,390,355. Examples of transparent titanium dioxide are commercially available under the tradenames TIPAQUE and TTO-51(A) from Ishihara Sangyo Kaisha, Ltd. (Japan). Another example of a transparent titanium dioxide is commercially available under the tradename T-COTE from BASF Corporation (Germany). Another example of transparent titanium dioxide is commercially available under the tradename UFTR from Miyoshi Kasei (Japan). Another example of transparent titanium dioxide is commercially available under the tradename SOLAVEIL CLARUS from Uniqema (Great Britain).

Preferably, the additional sunscreen agent is octyl methoxycinnamate, transparent zinc oxide, transparent titanium dioxide, or a mixture thereof.

In one embodiment, the additional sunscreen agents are present in an amount from about 0.1% to about 50% by weight of the composition, and more preferably 1% to about 25%.

Methylcellulose is generally available under the tradename METHOCEL A (The Dow Chemical Company). The polymeric backbone of cellulose is a repeating structure of anhydroglucose units. Treatment of cellulosic fibers with caustic solution, followed by methyl chloride, yields cellulose ethers substituted with methoxy groups. The term “DS” refers to the degree of methoxyl substitution per anhydroglucose unit. Preferably, the methylcellulose has a DS_{methoxyl} of about 1.47 to about 3.08.

In one embodiment, the methylcellulose has a viscosity at 2% concentration in water at 20° C., of about 1 cps to about 100,000 cps, preferably about 5 cps to about 30 cps, most preferably about 15 cps.

In one embodiment, the methylcellulose is present in an amount from about 0.01% to about 30% by weight of the composition, preferably from about 0.05% to about 15%, and more preferably greater than 1% to about 5%.

In one embodiment, the sunscreen composition is substantially free of hydroxypropyl methylcellulose.

In one embodiment, the sunscreen composition contains decyl glucoside.

In one embodiment, the sunscreen composition contains CORAPAN TQ Diethylhexyl 2,6 Naphthalate or another photostabilizer.

To make the sunscreen compositions, the organic pigment particulates are often added to the water phase.

Compositions of the present invention can further incorporate other ingredients known in the art of sunscreen formulations including at least one of cosmetically acceptable emollients, vitamins, moisturizers, conditioners, oils, silicones, suspending agents, opacifiers/pearlizers, surfactants, emulsifiers, preservatives, rheology modifiers, colorants, pH adjustors, propellants, reducing agents, anti-oxidants, fragrances, foaming or de-foaming agents, tanning agents, insect repellants, or biocides. Preferably, sunscreen compositions of the present invention include at least one of a humectant, a surfactant, an emollient, and a preservative.

In another embodiment, the present invention provides methods of boosting the SPF and/or PPD of a sunscreen composition having organic pigment particulates, the method comprising including methylcellulose in the sunscreen composition. In one embodiment, the sunscreen has an SPF that is at least about 25% higher, preferably at least about 50% higher, more preferably greater than about 50% higher, than sunscreens where methylcellulose is not present. In one embodiment, the sunscreen has an PPD that is at least about 25% higher, preferably at least about 50% higher, more preferably greater than about 50% higher, than sunscreens where methylcellulose is not present. In a preferred embodiment, both the SPF and the PPD are at least about 25% higher, preferably at least about 50% higher, more preferably greater than about 50% higher, than sunscreens where methylcellulose is not present.

### EXAMPLES

The following examples are for illustrative purposes only and are not intended to limit the scope of the present invention. All percentages are by weight unless otherwise specified.

### Example 1

Exemplary sunscreen compositions contain the components recited in TABLE 1.

TABLE 1

| | Component | Batch 1 |
|---|---|---|
| A | Deionized Water | 77.9 |
| | Propylene Glycol | 1.0 |
| | GLUCAM E-10 methyl gluceth-10 | 1.0 |
| | LIQUAPAR OPTIMA phenoxyethanol, | 0.9 |
| | methylparaben, isopropylparaben, | |
| | isobutylparaben, and butylparaben | |
| | METHOCEL A methylcellulose | 2.0 |
| | Xanthum Gum | 0.2 |
| | Bisoctrizole (50% dispersion) | 10.0 |
| B | PEG 40 stearate | 1.0 |
| | gylceryl stearate | 1.0 |
| | PROMULGEN D cetearyl alcohol and ceteareth-20 | 3.0 |
| | DC 200 Dimethicone | 2.0 |

METHOCEL A methylcellulose (DS 1.47-3.08; viscosity 10-20 cps at 2%) is slowly added to about 80° C. water. Once the methylcellulose is hydrated, the remaining Phase A (water) ingredients are added and the mixture maintained at 80° C. for about two hours.

The Phase B (oil) ingredients are combined in a separate container, heated to, and maintained at, about 80° C. for about two hours. Phase A and Phase B are then mixed and homogenized at about 11,000 rpm for about 60 seconds using a Silverson IKA Homogenizer.

### Example 2 (Comparative)

Comparative sunscreen compositions contain the components recited in TABLE 2.

TABLE 2

| | | Comparative |
|---|---|---|
| | Component | Batch A |
| A | Deionized Water | 79.9 |
| | Propylene Glycol | 1.0 |
| | GLUCAM E-10 methyl gluceth-10 | 1.0 |
| | LIQUAPAR OPTIMA phenoxyethanol, | 0.9 |
| | methylparaben, isopropylparaben, | |
| | isobutylparaben, and butylparaben | |
| | METHOCEL A methylcellulose | 0.0 |
| | Xanthum Gum | 0.2 |
| | Bisoctrizole (50% dispersion) | 10.0 |
| B | PEG 40 stearate | 1.0 |
| | gylceryl stearate | 1.0 |
| | PROMULGEN D cetearyl alcohol and ceteareth-20 | 3.0 |
| | DC 200 Dimethicone | 2.0 |

Comparative Batch A is prepared substantially as described above with respect to Batch 1, but without methylcellulose.

### Example 3

Sunscreen compositions prepared substantially according to the protocols of Examples 1 and 2 were prepared. Their SPF values determined and recited in TABLE 3A.

TABLE 3A

| | Batch 1 | Comparative Batch A |
|---|---|---|
| SPF | 20.9 ± 2.2_{—} | 12.5 ± _4.0_{—} |

The SPF was determined using an in vitro technique substantially according to the following protocol:

Initially, the weight of a roughened PMMA substrate (purchased from SCHÖNBERG GmbH & Co. KG, Hamburg/Germany,) is measured. The batch to be tested is then deposited on the substrate and then quickly leveled with a 7 micron draw down bar to achieve a thin, uniform layer. The layer is allowed to dry for about 20 minutes, and the weight of the substrate plus dry uniform layer is determined.

The UV absorption of dry uniform layer is measured using a LABSPHERE 1000S spectrometer at multiple points on the layer.

The percent solids of the layer is measured using a METTLER LP 16 solids analyzer. Using the weight of the dry film, and the solids content of the layer, the weight, and consequently the density of the original wet layer immediately after deposition can be calculated. Using this information, the SPF can be calculated by the following equation:

$$SPF = \frac{{\int }_{290 nm}^{400 nm} E ( λ ) S ( λ ) \partial λ}{{\int }_{290 nm}^{400 nm} E ( λ ) S ( λ ) {10}^{( - A ( λ ) )} \partial λ}$$

Where E(λ)=spectral irradiance of the Standard Sun Spectrum; S(λ)=erythemal action spectrum at wavelength λ; and A(λ)=corrected spectral absorbance at wavelength λ (a correction factor is calculated to extrapolate the data to establish what the absorbance would be at a wet layer density of 2.0 mg/cm² (using the original wet layer immediately after deposition).

The results in TABLE 3A are averages of at least seven measurements for each batch. The present invention results in SPF boosts of 67% as opposed to no methylcellulose.

For the PPD measurements, the SPF measurement procedure described above is used to obtain the absorption spectrum. The absorption spectrum is then analyzed using a method recommended by Colipa, the European Cosmetics Association, for measuring the PPD of sunscreens: Colipa UVA Method 2009. The PPD values determined are recited in TABLE 3B.

TABLE 3B

| | Batch 1 | Comparative Batch A |
|---|---|---|
| PPD | 19.4 ± 2.0 | 11.9 ± 3.6 |

The present invention results in PPD boosts of 63% as opposed to no methylcellulose.

It is understood that the present invention is not limited to the embodiments specifically disclosed and exemplified herein. Various modifications of the invention will be apparent to those skilled in the art. Such changes and modifications may be made without departing from the scope of the appended claims. Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A sunscreen composition, comprising:
organic pigment particulates; and
methylcellulose.

2. The sunscreen composition of claim 1, wherein the organic pigment particulates are bisoctrizole.

3. The sunscreen composition of claim 1, wherein the organic pigment particulates are present in an amount from about 0.1% to about 50% by weight of the composition, and more preferably 1% to about 25%.

4. The sunscreen composition of claim 1, wherein the methylcellulose is present in an amount from greater than 1% to about 5%.

5. The sunscreen composition of claim 1, wherein the methylcellulose has a DS_{methoxyl} of about 1.47 to about 3.08.

6. The sunscreen composition of claim 1, wherein the methylcellulose has a viscosity at 2% in water of about 1 cps to about 100,000 cps, preferably about 15 cps.

7. The sunscreen composition of claim 1, wherein sunscreen composition is substantially free of hydroxypropyl methylcellulose.

8. The sunscreen composition of claim 1, further comprising at least one additional sunscreen agent.

9. A method of boosting at least one of the SPF or PPD of a sunscreen composition comprising organic pigment particulates, comprising:
including methylcellulose in the sunscreen composition.

10. The method of claim 9, wherein the sunscreen has an SPF that is at least 25% higher than sunscreens where methylcellulose is not present.

11. The method of claim 9 or 10, wherein the sunscreen has a PPD that is at least 25% higher than sunscreens where methylcellulose is not present.
